(19) United States
(12) Patent Application Publication
(10) Pub. No.: US 2010/0168925 A1
(43) Pub. Date: Jul. 1, 2010

(54) DEVICE AND METHOD FOR CONTROLLING AN EXTRACORPOREAL BLOOD- TREATING APPARATUS

(76) Inventors: Peter Hilgers, Schonungen (DE); Jorg Jonas, Wehrheim (DE)

Correspondence Address: Kenyon & Kenyon LLP, One Broadway, New York, NY, 10004, US

(21) Appl. No.: 12/303,819

(22) PCT Filed: Jun. 6, 2007
(86) PCT No.: PCT/EP07/04993

§ 371 Date: Jul. 28, 2009

(30) Foreign Application Priority Data
Jun. 8, 2006 (DE) 10 2006 026 999.3
Aug. 17, 2006 (DE) 10 2006 038 545.4

(51) Int. Cl. G05D 7/06; A61M 1/14; G06F 17/10

(52) U.S. Cl. 700/282; 604/6.11; 703/2

(57) ABSTRACT

The present invention relates to a device and a method for controlling an extracorporeal blood treatment device, in particular a hemodialysis device that has a dialyzer, which is divided by a semi-permeable membrane into a blood chamber and a dialysis chamber, a blood pump for conveying blood through the blood chamber at a defined blood flowrate Q_{b}, and a dialysis pump for conveying dialysis fluid through the dialysis chamber at a defined dialysis flowrate Q_{d}. The control device and method according to the present invention for a hemodialysis device are based on the fact that, for different blood flow rates, in each case pre-defined during the blood treatment, the dialysis flowrates are determined at which a pre-defined clearance or dialysance is maintained with the pre-defined blood flowrates and/or that, for different dialysis flowrates in each case pre-defined during the blood treatment, the blood flowrates are determined at which the predefined clearance of dialysance is maintained.

### CROSS REFERENCE TO RELATED APPLICATIONS

This is a 371 national phase application of PCT/EP2007/004993 filed Jun. 6, 2007, claiming priority to German Patent Application No. 10 2006 026 999.3 filed Jun. 8, 2006, and German Patent Application No. 10 2006 038 545.4 filed Aug. 17, 2006.

### FIELD OF INVENTION

The present invention relates to an arrangement for controlling an extracorporeal blood-treating apparatus, and in particular a hemodialysis apparatus or hemofiltration apparatus or hemodiafiltration apparatus, which has a dialyzer or filter that is divided by a semi-permeable membrane into a first and a second chamber. The present invention also relates to an extracorporeal blood-treating apparatus having a control arrangement of this kind and to a method of controlling an extracorporeal blood-treating apparatus.

### BACKGROUND OF THE INVENTION

Hemodialysis is well known as an extracorporeal blood treatment process, in which the blood to be treated flows, in an extracorporeal blood circuit through the blood chamber of a dialyzer, which is divided by a semi-permeable membrane into a blood chamber and a dialysis fluid chamber, at a given blood flowrate, while dialysis fluid flows through a dialysis fluid chamber of the dialyzer at a given dialysis fluid flowrate. As well as hemodialysis, hemofiltration is also well known as a blood treatment process. Hemodiafiltration is a combination of both hemodialysis and hemofiltration.

The metabolic exchange in the dialyzer is of both a convective and a diffusive nature. In diffusive metabolic exchange, for the substance concerned, the mass transfer per unit of time through the membrane is proportional to the concentration gradient between the blood and the dialysis fluid; in convective metabolic exchange, the mass transfer depends on the quantity of filtrate because the concentration of filterable substances both in the blood and in the filtrate is the same (Blutreinigungsverfahren [Blood cleansing processes], Georg-Thieme-Verlag, Stuttgart, N.Y., 4th. ed. 1990, pages 11 to 13).

Because the concentration gradient becomes steadily smaller during the dialysis treatment, a fixed numerical value cannot be given for the amount of substance exchanged per unit of time. Clearance forms a measurable indicator of the performance of a dialyzer that is not dependent on concentration. The clearance of a substance is that part of the total flow through the dialyzer that has been entirely freed of the substance concerned. Dialysance is another term for defining the performance of a dialyzer, in a way which also takes substances that are contained in the dialysis fluid into account.

For ultrafiltration equal to 0, the following is found for the determination of dialysance D or clearance K for a given substance.

Dialysance D is equal to the ratio between the mass transport Q_{b}(c_{bi}−c_{bo}) on the blood side for the substance concerned and the difference c_{bi}−c_{di} in the concentration of the substance between the blood and the dialysis fluid at the given input of the dialyzer.

$$D = \frac{{Q}_{b} ( {c}_{bi} - {c}_{bo} )}{{c}_{bi} - {c}_{di}} \qquad ( 1 )$$

For reasons of mass balance, it is true that

Q_{b}(c_{bi}−c_{bo})=−Q_{d}(c_{di}−c_{do}) (2)

For dialysance on the dialysate side, the following equation follows from (1) and (2):

$$D ≡ - {Q}_{d} \frac{( {c}_{di} - {c}_{do} )}{{c}_{bi} - {c}_{di}} \qquad ( 3 )$$

The notation in (1) to (3) is as follows:

Q_{b}=effective blood flowrate
Q_{d}=dialysis fluid flowrate
C_{b}=concentration of the substance in the volume of the blood in which it is dissolved
C_{d}=concentration of the substance in the dialysis fluid
i=inlet of the dialyzer
o=outlet of the dialyzer.

EP 0 428 927 A1 describes a method of in vivo determination of parameters of hemodialysis in which dialysate electrolyte transfer is measured at each of two different dialysate input concentrations. On the assumption that the input concentration in the blood is constant, dialysance is determined by this known method of determining the difference between the differences in dialysis fluid ion concentration on the inlet side and outlet side of the dialyzer at the times of the first and second measurements, dividing this difference by the difference between the dialysis fluid ion concentrations on the input side at the times of the first measurement and the second measurement, and by multiplying the result by the flowrate of the dialysis fluid.

Another method of determining dialysance is described in U.S. Pat. No. 6,702,774 B1. In this known method, a given amount of a substance, whose dialysance is to be determined, is added upstream of the dialyzer as a bolus, and dialysance is calculated from the amount of the substance which is added upstream of the dialyzer, the integral of the concentration of the substance over time downstream of the dialyzer, and the flowrate of the dialysis fluid.

There is also a method of determining the maximum dialysance of a dialyzer which is known from DE 197 39 100 C1.

Sigdell and Tersteegen have studied the relationship between clearance and dialysance on the one hand, and blood and dialysis fluid flowrates on the other hand for dialysis without ultrafiltration (Sigdell, J., Tersteegen, B.: Clearance of a Dialyzer under varying Operation Conditions; Artificial Organs 10(3): 219-225, 1986). Sigdell and Tersteegen found that in practice, to increase clearance or dialysance, it appears not to make sense to set a dialysis fluid flowrate of more than twice the blood flowrate. Various methods have been proposed as ways of taking the effect of ultrafiltration on clearance into account. However, at the typical flowrates (Q_{f}=15 ml/min, Q_{d}=500 ml/min, Q_{b}=300 ml/min), the effect of ultrafiltration is relatively small and can be ignored. Werynski and Waniewski have found a general expression for the relationship between flowrates and the resultant clearance and have dealt with hemodiafiltration. They included hemodialysis as a special case (Wernyski, A. and Waniewski, J.: Theoretical Description of Mass Transport in Medical Membrane Devices, Artificial Organs 19(5), pp. 420-427 (1995)).

The known pieces of dialysis apparatus are operated at a blood flowrate which is set by the treating physician within predetermined limits, with the dialysis fluid flowrate likewise being set within predetermined limits, which are generally between 500 ml/min and 800 ml/min. This gives the dialysis dose, which is calculated from the quotient of the product of the clearance K and the effective treatment time T divided by the volume of distribution V (KT/V).

There is today a demand in practice for the quotient (K T/V) for urea to be higher than a pre-stipulated limiting value, and in particular to be higher than 1.3. The volume of distribution V depends on the patient in this case, which means that when treating the blood the physician can pre-stipulate only the clearance K, which is dependent on the flowrates of the blood and the dialysis fluid, and the treatment time T. Consequently, for the required dialysis dose to be achieved, there is a given value for clearance or dialysance which should be ensured to apply during a treatment and which can be found by calculation for a desired treatment time. If however the blood flowrate or the dialysis fluid flowrate changes during the treatment, it is not possible to ensure that a given clearance is obtained.

U.S. Pat. No. 5,092,836 describes a method of hemodialysis which is intended to allow a saving to be made of dialysis fluid. This method does not contemplate the pre-stipulation of a fixed value for the blood flowrate or the dialysis fluid flowrate. Instead, the intention is for a dialysis fluid flowrate to be pre-stipulated which is in a constant ratio to the pre-stipulated blood flowrate.

Also, there is known from WO 2004/022135 A1 a dialysis apparatus in which dialysance is measured and, by varying the rate of ultrafiltration, it is ensured that both the dialysis dose KT/V and the desired loss of weight by the patient are obtained at the same time.

U.S. Pat. No. 5,744,031 describes a method of controlling a blood treatment process in which, to determine dialysance, a measurement is made of conductivity, the value measured for dialysance being compared with a desired value to enable the blood flowrate or dialysis fluid flowrate to be altered in such a way that the actual value for dialysance corresponds to the desired value. This known method is disadvantageous inasmuch as a measurement of conductivity is required during the blood treatment to allow dialysance to be determined. However, a continuous measurement of conductivity not only involves greater cost and complication but also sets limits to how fast regulation can be, because a relatively large amount of time is required for the individual measurements if it is to be possible for the measured variables to be sensed with the requisite accuracy.

Both for hemodialysis and also for hemofiltration and a combination of the two processes, i.e. hemodiafiltration, the relationship between the flowrates on the one hand and clearance, and dialysance on the other hand is known from US 2003/0230533 A1, which is hereby incorporated by reference.

### SUMMARY OF THE INVENTION

One aspect of the present invention is to provide an arrangement for controlling an extracorporeal blood-treating apparatus which allows optimized blood treatment with a pre-stipulated clearance or dialysance. A further aspect of the present invention is to provide a blood-treating apparatus having such a control arrangement. Another aspect of the present invention is to specify a method of controlling an extracorporeal blood-treating apparatus which makes possible optimized blood treatment with a pre-stipulated clearance or dialysance. A further aspect of the present invention is to provide a computer software product for such a control arrangement.

The control arrangement according to the present invention and the method according to the present invention are intended for an extracorporeal blood-treating apparatus which make take the form both of a hemodialysis apparatus and of a hemofiltration apparatus. The control arrangement according to the present invention and the method may also be intended for a hemodiafiltration apparatus.

The different instances of application differ in that different flowrates, which each have an effect on dialysance or clearance, play a part in the individual treatment processes. In this way, provision is made in hemodiafiltration not only for changing the blood flowrate and the dialysis fluid flowrate but also for changing the ultrafiltration flowrate or the substituent flowrate. However, since the dependence of dialysance or clearance on the individual flow rates is known for all the instances of application, there is no fundamental difference between the alternative embodiments of the control arrangement according to the present invention.

In the general case of extracorporeal blood treatment which covers all the treatment processes, what will be referred to will be an exchanging unit, which may take the form either of a dialyzer or a filter in the case of hemodialysis or hemofiltration, respectively. The extracorporeal hemodialysis apparatus for example, to which one embodiment relates, has a dialyzer, which is divided by a semi-permeable membrane into a blood chamber and a dialysis fluid chamber, a blood pump for pumping blood through the blood chamber at a given blood flowrate Q_{b}, and a dialysis fluid pump for pumping dialysis fluid through the dialysis fluid chamber at a given dialysis fluid flowrate Q_{d}.

The control arrangement according to the present invention may form an independent assembly or may be part of the extracorporeal blood-treating apparatus. Because major components of the control arrangement according to the present invention, such for example as a control unit (microprocessor) and a memory unit, are parts of the known blood-treating apparatus, the control arrangement according to the present invention can be provided in the known blood-treating apparatus at no great technical cost or complication. If all the hardware required is available, the provision of the computer software product according to the present invention may be all that is required.

The arrangement and method according to the present invention assume that different flows or flowrates are pre-stipulated before the treatment, and/or different flows or flowrates, such as blood flowrates or dialysis fluid flowrates, are altered during the treatment. When a blood flowrate, for example, is pre-stipulated or changed respectively before or during the blood treatment, the dialysis fluid flowrate is pre-stipulated or changed in such a way that a desired clearance or dialysance is maintained, preferably for a pre-stipulated period of treatment. Basically, the dialysis fluid flowrate may only be pre-stipulated but need not be set automatically. Preferably however the dialysis fluid flowrate at which the desired clearance or dialysance is maintained is also set by the apparatus. This may be done automatically or after confirmation by a user.

The blood flowrate may be changed during the blood treatment once or more than once but basically even continuously, the dialysis fluid flowrate then always being set in such a way that the desired clearance or dialysance is maintained, preferably within the pre-stipulated period of treatment. Conversely, when there is a change in the dialysis fluid flowrate the blood flowrate is set such that the desired clearance or dialysance is maintained, preferably within the pre-stipulated period of treatment. What is crucial is that the flowrate in the given case is determined not on the basis of a measurement of clearance or dialysance, such as a measurement of conductivity for example, but on the basis of the known dependence of clearance or dialysance on the flowrates. In this way it is possible for the flowrates to be adjusted quickly and continuously to ensure that clearance or dialysance is as desired during the treatment.

Basically, both the dialysis fluid flowrate and also the blood flowrate may be changed. In practice however, the blood flowrate is generally changed and then the dialysis fluid flowrate is only adjusted to suit.

The desired clearance or dialysance is preferably entered prior to the blood treatment from an input unit and is stored in a memory unit. The desired dialysance or clearance thus constitutes a desired value (i.e. a setpoint) for the control system.

A further possible embodiment makes provision not for entering desired values for clearance or dialysance, but for the measurement of these parameters. Clearance or dialysance is preferably measured at the beginning of the dialysis treatment, with control then taking place over the course of the treatment without any further measurement of dialysance or clearance. The measured value thus constitutes the desired value that is to be achieved by the blood treatment even if the dialysis fluid flowrate or the blood flowrate is changed during the treatment. The actual methods of measuring dialysance or clearance are part of the prior art.

The relationship between the desired clearance or dialysance on the one hand and, for example, the blood flowrate or dialysis fluid flowrate on the other hand, can be defined by an equation. This equation includes only the parameters of blood flowrate and dialysis fluid flowrate and a coefficient k0A, which is dependent in essence on the surface area and on the resistance to diffusion of the semi-permeable membrane of the dialyzer. This coefficient k0A can be pre-stipulated and stored for various types of dialyzers before the blood treatment begins. It is however also possible for the coefficient k0A to be determined by measuring the clearance or dialysance at a pre-stipulated blood flowrate and dialysis fluid flowrate and calculating the coefficient from an equation which defines the relationship between clearance or dialysance and between blood flowrate and dialysis fluid flowrate.

### BRIEF DESCRIPTION OF THE DRAWINGS

An embodiment of the present invention is described in detail below by reference to the drawings.

FIG. 1 is a simplified schematic view of the conditions which apply to the liquids or fluids in hemodialysis.

FIG. 2a is a simplified schematic view of the conditions which apply to the liquids or fluids in hemofiltration when there is predilution.

FIG. 2b is a simplified schematic view of the conditions which apply to the liquids or fluids in hemofiltration when there is postdilution.

FIG. 3 is a simplified schematic view of the main components of an extracorporeal blood-treating apparatus according to the present invention, together with a control arrangement according to the present invention, in the case of hemodialysis.

### DETAILED DESCRIPTION OF THE DRAWINGS

The relationship in extracorporeal blood treatment between dialysance or clearance and the flowrates will be explained below. The dependence of dialysance or clearance on the flowrates is described in detail in US 2003/0230533 A1, the disclosure of which is hereby incorporated by reference.

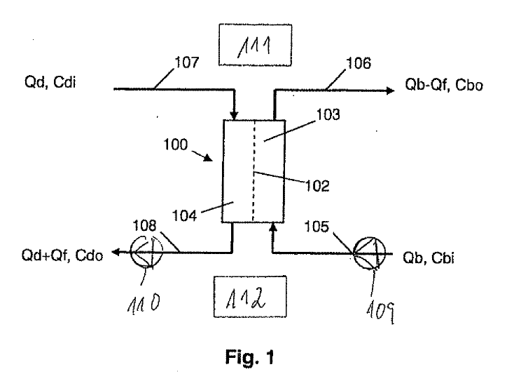

FIG. 1 shows a hemodialysis treatment embodiment. The hemodialyzer 100 is divided by a semi-permeable membrane 102 into two chambers 103 and 104, with fresh dialysis fluid flowing into the first chamber 104 from a dialysis fluid inlet line 107 at a flowrate Q_{d} and with a physicochemical attribute C_{di}. From this chamber 104, a flow Q_{d}+Q_{f} of dialysis fluid, which has been increased by the ultrafiltration flow Q_{f} needing to be removed and which has the physicochemical attribute C_{do}, flows away via a dialysis fluid outlet line 108. Blood flows into the second chamber 103 from a blood inlet line 105 at a flowrate Q_{b} and with a physicochemical attribute C_{bi}. A flow of blood that has been reduced by the ultrafiltration flow Q_{f} and that has the physicochemical attribute C_{bo} leaves this chamber 103 by way of the blood outlet line 106. The blood is pumped by a blood pump 109 and the dialysis fluid is pumped by a dialysis fluid pump 110, the pumping rates which determine the blood flowrate and the dialysis fluid flowrate, respectively. The ultrafiltration flowrate is pre-stipulated by an ultrafiltration arrangement which is identified as 111. A control unit 112 which has a calculating unit is responsible for monitoring the blood treatment and for setting the respective flowrates.

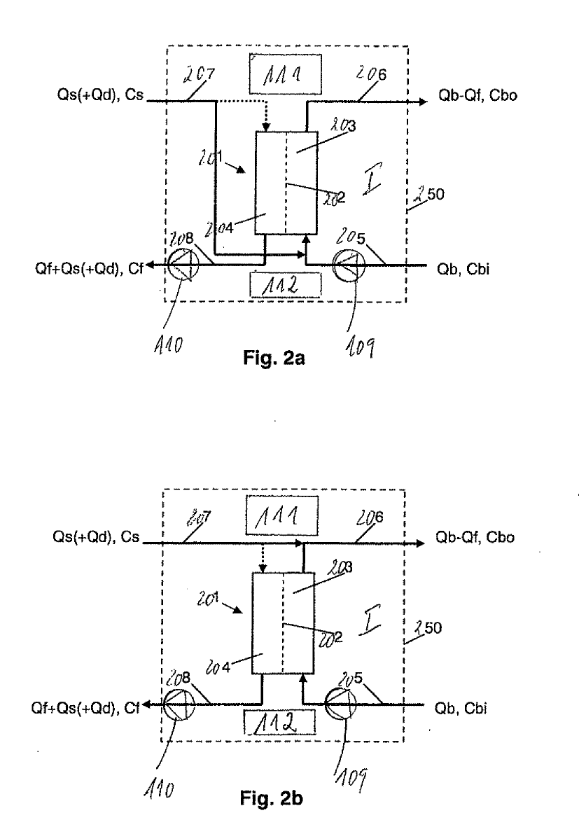

FIGS. 2a and 2b are similarly schematic views of a hemofiltration apparatus in which a hemofilter 201, which is divided by a semi-permeable membrane 202 into two chambers 203 and 204, is provided as an exchanging unit. On the blood side, the terminology that applies is the same as in FIG. 1. Also provided is an substituent fluid inlet line 207 that is directly connected either to the blood inlet line 205 (predilution, FIG. 2a) or to the blood outlet line 206 (postdilution, FIG. 2b). Through this line 207, substituent fluid is fed directly, i.e. not via the membrane 202, to the extracorporeal blood circuit T at a flowrate Qₛ and with a physicochemical attribute Cₛ. Furthermore, fluid is withdrawn from the blood, via the membrane 202, at a flowrate Qₒ=Q_{f}+Qₛ, which fluid flows into the first chamber 204 and leaves this chamber via the ultrafiltrate outlet line 208 with a physicochemical attribute C_{f}. The ultrafiltration arrangement and the control unit are once again identified by reference numerals 111 and 112, respectively.

Also shown in FIGS. 2a and 2b is a path indicated as a dashed line which branches off from the substituent fluid inlet line 207 and runs to the first chamber 204. In a hemodiafiltration application, there is flow along this path as well. The conditions of flow thus change inasmuch as the terms which are shown in brackets come into play in addition for the dialysis fluid flowrate Q_{d}. The flow along the ultrafiltrate takeaway line then comes to Qₒ=Q_{f}+Qₛ+Q_{d}. The same references Cₛ and C_{f} are still used for the physicochemical attributes. For the path shown in FIGS. 2a and 2b, Cₛ remains unchanged by the hemodiafiltration. The value of C_{f} however will change because the proportion Q_{d} of the flow Qₛ+Q_{d} having the physicochemical attribute Cₛ now flows through the first chamber 204 and mixes with the flow Qₛ+Q_{f} which is added to it through the membrane 202, both to be taken away together by the ultrafiltrate outlet line 208.

The relationship between the flowrates and dialysance will be described below. It is assumed that a given dialysance and at least one of the flowrates are pre-stipulated to allow at least one of the other flowrates to be calculated so that the desired dialysance will be maintained. The relationship which is detailed below covers the case where a given dialysance and at least one of the flowrates is pre-stipulated during the treatment. If a given dialysance is pre-stipulated during the treatment, the relevant flowrates can be calculated, thus allowing the desired dialysance to be maintained. This calculation may be made whenever one of the flowrates has changed, without the need for a conductivity measurement.

The diffusive proportion D_{diff} of the dialysance is calculated from equation (4):

$${D}_{diff} ≡ ( \frac{{Q}_{b} + {kQ}_{d}}{{Q}_{b} - {Q}_{f} ( 1 - k ) {Q}_{s}} ) ( \frac{{Q}_{b} + {kQ}_{s}}{{Q}_{b}} D - {Q}_{f} - {Q}_{s} ) \qquad ( 4 )$$

where k=1 is predilution and k=0 is postdilution.

$$k 0 A = \frac{( {Q}_{b} + {kQ}_{s} ) {Q}_{d}}{{Q}_{d} - {Q}_{b} - {kQ}_{s}} In \frac{\frac{{D}_{diff}}{{Q}_{d}} - 1}{\frac{{D}_{diff}}{{Q}_{b} + {kQ}_{s} - 1}} \qquad ( 5 )$$

The filter coefficient k0A, which is assumed to be constant between the two points in time 1 and 2, is calculated as follows:

$${D}_{diff} = Qb \frac{{e}^{ γ} - 1}{{e}^{ γ} - \frac{{Q}_{b}}{{Q}_{d}}} , where γ = k 0 A \frac{{Q}_{d} - {Q}_{b}}{{Q}_{b} {Q}_{d}} \qquad ( 6 )$$

The above equations show the relationship between the flowrates Q_{f}, Qₛ, Q_{d} and Q_{b} and the parameters k0A, D and D_{diff}.

In accordance with the present invention, at least one of the flowrates is pre-stipulated, with at least one of the other flowrates being determined from the above equations, thus allowing the desired dialysance D to be obtained. The determination of the flowrates from the above equations can be performed numerically by known methods of calculation.

In what follows, a method of determining dialysance D which is known from US 2003/0230533 will be described in brief by reference to FIGS. 2a and 2b. A region 250 is outlined by a dashed line in FIGS. 2a and 2b. If this region is considered to be a sort of black box constituting a dialyzer 1, then the formal provisions which apply to the arrangement shown in FIG. 1 can be transferred to the situation which exists in hemofiltration. If the physicochemical attribute is a concentration, the equations found are as follows:

$$D = ( {Q}_{f} + {Q}_{s} + {Q}_{d} ) \frac{{C}_{f} - {C}_{s}}{α {C}_{bi} - {C}_{s}} \qquad ( 7 )$$

Where α is the Gibbs-Donnan coefficient.

$$\begin{matrix}D = ( {Q}_{f} + {Q}_{s} + {Q}_{d} ) ( 1 - \frac{{C}_{f 2} - {C}_{f 1}}{{C}_{s 2} - {C}_{s 1}} ) \\ = ( {Q}_{f} + {Q}_{s} + {Q}_{d} ) ( 1 - \frac{Δ {C}_{f}}{Δ {C}_{s}} )\end{matrix} \qquad ( 8 )$$

After an initial determination of the ion dialysance D, it is possible for further values of dialysance to be calculated for later points in time at which at least one of the flowrates Qₛ, Q_{f}, Q_{d} and Q_{b} has changed. However, this presupposes that the flowrates Qₛ, Q_{f}, Q_{d} and Q_{b} are known at a time before they change. The initial determinations of dialysance at the flowrates Q_{f1}, Qₛ₁, Q_{d1} and Q_{b1} can be performed by known methods on the basis of a measurement of conductivity. These methods are part of the prior art and thus there is no need for any further description of them. A method of this kind is described in, for example, EP 0 428 927 A1 or US 2003/0230533.

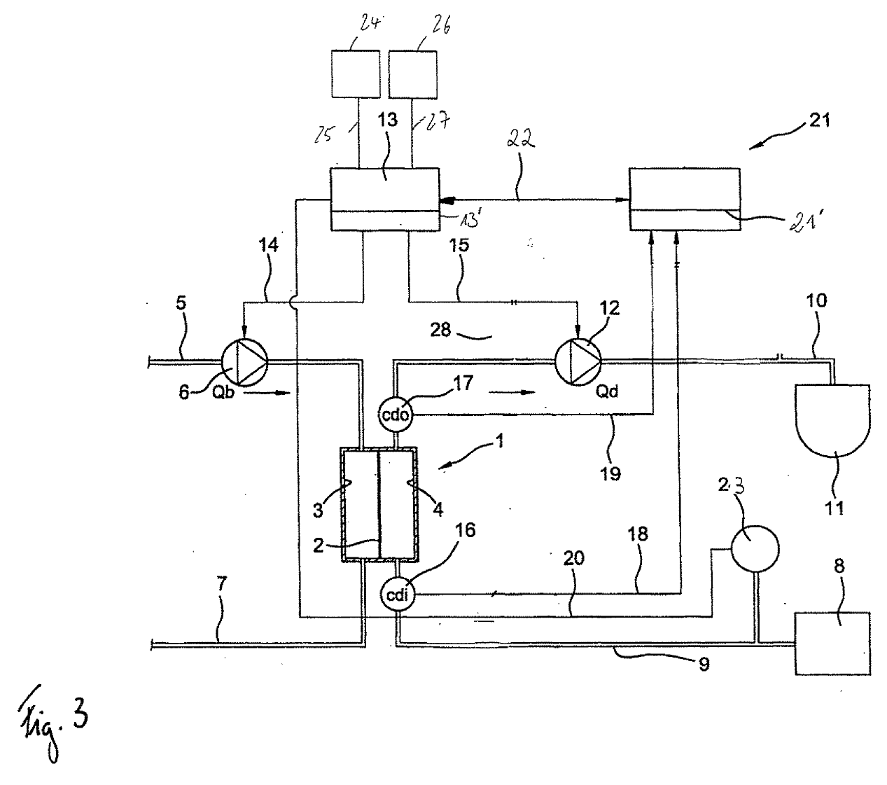

In what follows, the relationship described above between clearance or dialysance and the individual flows, i.e. flowrates, will be explained by reference to FIG. 3 as it applies to the case of hemodialysis.

The extracorporeal blood-treating apparatus, which is a hemodialysis apparatus, has a dialyzer 1 which is divided by a semi-permeable membrane 2 into a blood chamber 3 and a dialysis fluid chamber 4. From a patient, an arterial blood line 5, into which a blood pump 6 is connected, runs to an inlet of the blood chamber 3, while a venous blood line 7 runs to the patient from an outlet of the blood chamber 3.

Fresh dialysis fluid is supplied from a source 8 of dialysis fluid. From the source 8 of dialysis fluid, a dialysis fluid inlet line 9 runs to an inlet of the dialysis fluid chamber 4 of the dialyzer 1, while a dialysis fluid outlet line 10 runs from an outlet of the dialysis fluid chamber 4 to a discharge outlet 11. Connected into the dialysis fluid outlet line 10 is a dialysis fluid pump 12.

The dialysis apparatus has a control unit 13 which is connected to the blood pump 6 and the dialysis fluid pump 12 by control lines 14, 15, respectively. The control unit 13 generates control signals for operating the blood and dialysis fluid pumps 6, 12 at pre-stipulated pumping rates, which means that a pre-stipulated blood flowrate Q_{b} is established in the arterial blood line 5 and a pre-stipulated dialysis fluid flowrate Q_{d} is established in the dialysis fluid outlet line 10.

Arranged in the dialysis fluid inlet line 9, at the inlet of the dialysis fluid chamber 4, are a conductivity sensor 16 for determining the input concentration c_{d}, of a given substance in the dialysis fluid upstream of the dialysis fluid chamber 4 and, in the dialysis fluid outlet line 10, at the outlet of the dialysis fluid chamber 4, a conductivity sensor 17 which measures the output concentration c_{do} of the given substance in the dialysis fluid downstream of the dialyzer, during the dialysis treatment.

The measured values from the conductivity sensors 16, 17 are fed via signal lines 18, 19, respectively, to an arrangement 21 for determining the clearance K or dialysance D. Via a data line 22 that runs to the control unit 13, the arrangement 21 for determining clearance or dialysance receives the control signals for the blood pump 6 and dialysis fluid pump 12 which pre-stipulate the blood flowrate Q_{b}, and dialysis fluid flowrate Q_{d} respectively. From the arrangement 21, the control unit 13 receives, via the data line 22, the clearance or dialysance that is determined by the arrangement 21.

Arrangement 23 is provided to allow the concentration of Na in the dialysis fluid upstream of the dialyzer 1 to be changed. Via a control line 20, the arrangement 23 is connected to the control unit 13.

An input unit 24 is also connected to the control unit 13 by a data line 25. A desired clearance K or dialysance D can be entered from the input unit 13. It is also possible for a desired blood flowrate Q_{b} or dialysis fluid flowrate Q_{d} to be entered to enable either one or both of the parameters to be pre-stipulated and/or to be changed during the treatment. Also provided is a memory unit 26 which is connected to the control unit 13 by data line 27. The values entered from the input unit 24 are stored in the memory unit 26 and can be read from the memory unit 26 by the control unit 13.

The dialysis apparatus permits various modes of operation which will be described in detail below. However, it is not a prerequisite of all the modes of operation for dialysance or clearance to be measured. Therefore, the arrangement for measuring clearance or dialysance which is formed by the components identified by reference numerals 21, 23, 16 and 17 can also be dispensed with for the relevant modes of operation.

The dialysis apparatus also has other components, e.g. a drip chamber, shut-off members, etc. which are known to the person skilled in the art but which have not been shown for the sake of greater clarity. The dialysis apparatus may also have an ultrafiltration arrangement.

Using the input unit 24, which has, for example, screen input facilities or a keyboard, the user enters the desired clearance K or dialysance D as well as various other parameters for the hemodialysis. It is also possible for the duration T of the treatment and a desired blood flowrate Q_{b} and/or dialysis fluid flowrate Q_{d} to be entered. The values are stored in the memory unit 26 and can be read off by the control unit 13.

The control unit 13 has a calculating unit 13′ which, from the desired clearance or dialysance and the blood flowrate, calculates that dialysis fluid flowrate that is required to enable the desired clearance or dialysance to be achieved. If what the user pre-stipulated was not the blood flowrate but the dialysis fluid flowrate, then the calculating unit 13′ would calculate the blood flowrate that is required to enable the desired clearance or dialysance to be achieved.

Using the coefficient k0A, the calculation of the required dialysis fluid flowrate Q_{b} or blood flowrate Q_{d} is performed on the basis of the following equation:

$$K = {Q}_{b} {Q}_{d} \frac{1 - exp ( - k 0 A \frac{{Q}_{d} - {Q}_{b}}{{Q}_{d} {Q}_{b}} )}{{Q}_{d} - {Q}_{b} exp ( k 0 A \frac{{Q}_{d} {Q}_{b}}{{Q}_{d} {Q}_{b}} )} \qquad ( 9 )$$

In this equation, k0A is a coefficient that depends in essence on the active surface area A of the semi-permeable membrane of the dialyzer (in m²) and on the resistance to diffusion R of the membrane of the dialyzer (in m² min/ml=10⁴ min/cm) (k0A=A/R).

Whereas the equations given previously define the relationship in which dialysance or clearance stands in a general form, equation (9) defines this relationship for the special case of hemodialysis. Numerical methods which are familiar to the person skilled in the art are generally employed to solve the equation.

The coefficient k0A is a characteristic typical of the dialyzer which is read from the memory unit 26 by the control unit 13. A plurality of coefficients k0A which are associated with different types of dialyzers may be stored in the memory unit 26. Using the input unit 24, the user is able to enter a given type of dialyzer before the treatment begins, thus enabling the control unit 13 to read the coefficient that applies in the given case from the memory unit 26.

Due to the relationship given in equation (9) between blood flowrate and also dialysis fluid flowrate and clearance or dialysance, a reduction in blood flowrate leads to an increase in dialysis fluid flowrate if the desired clearance or dialysance is to be achieved. Conversely, an increase in blood flowrate leads to a reduction in dialysis fluid flowrate if the pre-stipulated clearance or dialysance is to be achieved. If on the other hand it is not the blood flowrate but the dialysis fluid flowrate which is changed, then an increase in dialysis fluid flowrate leads to a reduction in blood flowrate and a reduction in dialysis fluid flowrate leads to an increase in blood flowrate.

If a change is made to the dialysis fluid flowrate or blood flowrate during the treatment, which change may be made in individual steps or continuously, the blood flowrate or dialysis fluid flowrate, in the respective cases, is always adjusted by the control unit 13 such that the desired clearance or dialysance is maintained over the pre-stipulated treatment time.

When controlling the pumping rate of the blood pump 6 and dialysis fluid pump 12, the control unit 13 takes account of the fact that for the blood flowrate and dialysis fluid flowrate there are certain respective minimum or maximum flowrates which must not be dropped below or exceeded. The blood flowrate in particular should not exceed a certain upper limiting value which depends on the vascular access. In the event that the achieving of the desired clearance or dialysance should make it necessary for the respective flowrates for the blood or the dialysis fluid to be exceeded or dropped below, the control unit 13 signals this fault condition to the user. What may be provided for this purpose is for example an alarm arrangement (not shown) which gives an audio and/or visual alarm. The control unit 13 can then pre-stipulate a longer or a shorter treatment time to enable a setting to be made for the flowrate that is within the pre-stipulated limits for blood flowrate and dialysis fluid flowrate.

In what follows, an alternative embodiment of the control arrangement will be described which makes use of the arrangement for measuring dialysance.

The basis for the measurement of dialysance is that the input concentration c_{di}, such as, for example, as the concentration of Na in the dialysis fluid, upstream of the dialyzer 1 is changed for a short time by the arrangement 23 for changing the composition of the dialysis fluid and the input concentration c_{di} and output concentration c_{d}, in the dialysis fluid are measured upstream and downstream of the dialyzer by the conductivity sensors 16, 17, respectively. The values measured are processed by the arrangement 21 for determining clearance or dialysance, which has a calculating unit 21′ to calculate the clearance or dialysance.

The calculation of clearance K or dialysance D for a given blood flowrate and dialysis fluid flowrate can be performed using equations (1) to (3). This method of determining clearance or dialysance is described in detail in EP 0 428 927 A1, which is hereby incorporated by reference.

A further method of determining clearance or dialysance, which is distinguished by having a particularly short measuring time, is known from U.S. Pat. No. 6,702,774 B1, which is likewise hereby incorporated by reference. Due to the short measuring times, the application of this method is given preference in the case of the control arrangement according to the present invention.

The control unit 13 controls the arrangement 21 for determining clearance or dialysance at the beginning of the treatment, and the arrangement 21 thus determines clearance or dialysance at the blood flowrate and dialysis fluid flowrate which are pre-stipulated at the beginning of the treatment. The value determined for clearance or dialysance is then read off by the control unit, which calculates the coefficient k0A on the basis of equation (9), which is then available for the continued calculation of the flowrates from equation (9). This embodiment has the advantage that the type of dialyzer does not have to be entered from the input unit 24 and there does not have to be a table in which different coefficients are assigned to different types of dialyzers stored in the memory unit 26.

A further alternative embodiment provides for the desired clearance or dialysance not to be entered from the input unit 24, but rather to be pre-stipulated by the control unit 13 at the beginning of the treatment. What the control unit 13 may pre-stipulate as clearance or dialysance is the value that the arrangement 21 for determining clearance or dialysance determined at, for example, the beginning of the treatment.

The formal provisions which have been described by reference to hemodialysis can also be applied to hemofiltration. The control arrangement for a hemofiltration apparatus therefore differs from the control arrangement described above only in that, as well as the blood flowrate, account is also taken of the ultrafiltration flowrate and/or the substituent flowrate in the evaluation of the flowrates, but account is not taken of the dialysis fluid flowrate. In hemodiafiltration, account is taken not only of the blood flowrate and the dialysis fluid flowrate but also of the ultrafiltration flowrate and/or the substituent flowrate. If one of the flowrates, such as the dialysis fluid flowrate for example, is altered, the calculating unit 13′ of the control unit 13 calculates, on the basis of the relationship defined in equations (4) to (6), one of the other flowrates, such for example as the blood flowrate or ultrafiltration flowrate or substituent flowrate, at which there is an assurance of the desired clearance or dialysance being achieved during the blood treatment, but without the need for measurements of conductivity to be made continuously. All that is required in this case is for clearance or dialysance to be measured once for a set of flowrates Q_{f1} and/or Qₛ₁ and/or Q_{d1} and/or Q_{b1}, so that, if there is a change in a flowrate, another flowrate can be adjusted in the appropriate way simply on the basis of a calculation of the parameters. If this measurement is made by the arrangement 21 for determining clearance or dialysance, by making the measurement of conductivity after a brief change in the composition of the dialysis fluid or substituent fluid. The calculation of whichever is the other flowrate, which is intended to counteract the alteration in the one flowrate in order to ensure that the pre-stipulated clearance or dialysance is achieved, will be made whenever the one flowrate has been altered.

1-27. (canceled)

28. A system for controlling a hemodialysis apparatus, comprising:
a dialyzer divided by a semi-permeable membrane into a blood chamber and a dialysis fluid chamber;
a blood pump for pumping blood through the blood chamber at a blood flowrate Q_{b};
a dialysis fluid pump for pumping dialysis fluid through the dialysis fluid chamber at a dialysis fluid flowrate Q_{d};
a memory unit for storing a desired clearance K or dialysance D; and
a control unit for setting the blood pump to a blood flowrate Q_{b} and setting the dialysis fluid pump to a dialysis fluid flowrate Q_{d}, the control unit comprising:
a calculating unit configured to receive an initial blood flowrate Qb, and a desired clearance K or dialysance D, and calculate the dialysis fluid flowrate Qd to be set therefrom, or to receive an initial dialysis fluid flowrate Qd, and a desired clearance or dialysance, and calculate the blood flowrate Qb to be set therefrom.

29. The system of claim 28, wherein the calculating unit is adapted to calculate the blood flowrate Q_{b} or dialysis fluid flowrate Q_{d} continuously if there is a change in the initial dialysis fluid flowrate Q_{d} or initial blood flowrate Q_{b}.

30. The system of claim 28, further comprising an input unit for entering the desired clearance K or dialysance D, and cooperating with the memory unit to store the desired clearance K or dialysance D.

31. The system of claim 30, wherein the input unit is also adapted to enter at least one of a desired blood flowrate and a desired dialysis fluid flowrate into the control unit, and the control unit is adapted to operate at least one of the blood pump and the dialysate fluid pump at the entered flowrates.

32. The system of claim 28, wherein the calculating unit is designed such that if there is an increase in the blood flowrate Q_{b}, then the dialysis fluid flowrate Q_{d} will be decreased sufficiently, and if there is a decrease in the blood flowrate Q_{b}, then the dialysis fluid flowrate Q_{d} will be increased sufficiently, and/or if there is an increase in the dialysis fluid flowrate Q_{d}, then the blood flowrate Q_{b} will be reduced sufficiently, and if there is a reduction in the dialysis fluid flowrate Q_{d}, then the blood flowrate Q_{b} will be increased sufficiently, for the pre-stipulated clearance or dialysance to be maintained.

33. The system of claim 28, wherein the relationship between the desired clearance K or dialysance D and the blood flowrate Q_{b} and dialysis fluid flowrate Q_{d} is defined by the following equation:
$$K = {Q}_{b} {Q}_{d} \frac{1 - exp ( - k 0 A \frac{{Q}_{d} - {Q}_{b}}{{Q}_{d} {Q}_{b}} )}{{Q}_{d} - {Q}_{b} exp ( k 0 A \frac{{Q}_{d} {Q}_{b}}{{Q}_{d} {Q}_{b}} )}$$
where k0A is a coefficient.

34. The system of claim 33, wherein different values for the coefficient k0A are stored in the memory unit for different types of dialyzers, and the proper value for the dialyzer used is transmitted to the control unit.

35. The system of claim 28, further comprising a measuring unit for measuring the clearance K or dialysance D, and transmitting the measured clearance K or dialysance D to the control unit to calculate the coefficient k0A for the initial blood flowrate Q_{b} and the initial dialysis fluid flowrate Q_{d}.

36. A system for controlling an extracorporeal blood-treating apparatus comprising:
an exchanging unit divided by a semi-permeable membrane into a first chamber and a second chamber, wherein the first chamber is part of an extracorporeal blood circuit comprising a blood pump for pumping blood at a blood flowrate Q_{b}, and the second chamber is part of a dialysis circuit comprising a dialysis pump for pumping dialysis fluid at a dialysis fluid flowrate Q_{d};
at least one of a substituent inlet line for feeding substituent directly to the extracorporeal blood circuit having a substituent flowrate Qₛ, and an ultrafiltrate outlet line from the first chamber having a flowrate that corresponds to the sum of the substituent flowrate Qₛ and an ultrafiltration flowrate Q_{f};
a memory unit for storing a desired clearance K or dialysance D; and
a control unit for setting the blood pump to a blood flowrate Q_{b} and for setting at least one of the dialysis fluid flowrate Q_{d}, the ultrafiltration flowrate Q_{f}, or the substituent flowrate Qₛ, the control unit comprising:
a calculating unit configured to receive a desired clearance K or dialysance D and at least one first flowrate chosen from the group consisting of: blood flowrate Q_{b}, dialysis fluid flowrate Q_{d}, ultrafiltrate flowrate Q_{f}, and substituent flowrate Qs, and calculate at least one second flowrate from one of the other flowrates chosen from the group consisting of: blood flowrate Q_{b}, dialysis fluid flowrate Q_{d}, ultrafiltrate flowrate Q_{f}, and substituent flowrate Qₛ, wherein the desired clearance K or dialysance D is maintained.

37. The system of claim 28, wherein the calculating unit is adapted to calculate the at least one second flowrate continuously if there is a change in the at least one first flowrate set by the control unit.

38. A method of controlling an extra-corporeal blood-treating apparatus, the extra-corporeal blood-treating apparatus comprising:
an exchanging unit divided by a semi-permeable membrane into a first chamber and a second chamber, wherein the first chamber is part of an extracorporeal blood circuit comprising a blood pump for pumping blood at a blood flowrate Q_{b}, and the second chamber is part of a dialysis circuit comprising a dialysis pump for pumping dialysis fluid at a dialysis fluid flowrate Q_{d};
at least one of a substituent inlet line for feeding substituent directly to the extracorporeal blood circuit having a substituent flowrate Qₛ; and an ultrafiltrate outlet line from the first chamber having a flowrate that corresponds to the sum of the substituent flowrate Qₛ and an ultrafiltration flowrate Q_{f};
the method comprising the following steps:
storing a desired clearance K or dialysance D,
setting at least one first flowrate chosen from the group consisting of blood flowrate Q_{b}, dialysis fluid flowrate Q_{d}, ultrafiltrate flowrate Q_{f} and substituent flowrate Qₛ; and
calculating at least one second flowrate of one of the other flowrates chosen from the group consisting of: blood flowrate Q_{b}, dialysis fluid flowrate Q_{d}, ultrafiltrate flowrate Q_{f} and substituent flowrate Qₛ.

39. The method of claim 38, further comprising calculating the at least one second flowrate continuously, if there is a change in the at least one first flowrate.

40. The method of claim 38, further comprising setting the at least one second flowrate after calculation thereof.

41. A method of controlling a hemodialysis apparatus, the hemodialysis apparatus comprising:
a dialyzer divided by a semi-permeable membrane into a blood chamber and a dialysis fluid chamber;
a blood pump for pumping blood through the blood chamber at a blood flowrate Q_{b};
a dialysis fluid pump for pumping dialysis fluid through the dialysis fluid chamber at a dialysis fluid flowrate Q_{d};
the method comprising the following steps:
storing a desired clearance K or dialysance D; and
for an initial blood flowrate Q_{b} and the desired clearance K or dialysance D, calculating the dialysis fluid flowrate Q_{d} to be set; or
for an initial dialysis fluid flowrate Q_{d} and the desired clearance K or dialysance D, calculating the blood flowrate Q_{b} to be set.

42. The method of claim 41, further comprising calculating continuously the blood flowrate Q_{b} or dialysis fluid flowrate Q_{d}, if there is a change in the initial dialysis fluid flowrate Q_{d} or initial blood flowrate Q_{b}.

43. The method of claim 41, further comprising setting the dialysis fluid flowrate Q_{d} or blood flowrate Q_{b} that is calculated.

44. The method of claim 41, further comprising entering at least one of a desired blood flowrate Q_{b} and a dialysis fluid flowrate Q_{d}, and operating the blood pump or dialysis fluid pump at a pumping rate such that the desired blood flowrate or dialysis fluid flowrate is established.

45. The method of claim 41, further comprising, maintaining the desired clearance or dialysance D by:
sufficiently decreasing the dialysis fluid flowrate Q_{d} if there is an increase in the blood flowrate Q_{b},
sufficiently increasing the dialysis fluid flowrate Q_{d} if there is a reduction in the blood flowrate Q_{b},
sufficiently decreasing the blood flowrate Q_{b} if there is an increase in the dialysis fluid flowrate Q_{d}, and
sufficiently increasing the blood flowrate Q_{b} if there is a reduction in the dialysis fluid flowrate Q_{d}.

46. The method of claim 45 wherein the relationship between the desired clearance K or dialysance D and the blood flowrate Q_{b} and dialysis fluid flowrate Q_{d} is defined by the following equation:
$$K = {Q}_{b} {Q}_{d} \frac{1 - exp ( - k 0 A \frac{{Q}_{d} - {Q}_{b}}{{Q}_{d} {Q}_{b}} )}{{Q}_{d} - {Q}_{b} exp ( k 0 A \frac{{Q}_{d} {Q}_{b}}{{Q}_{d} {Q}_{b}} )}$$
where k0A is a coefficient.

47. The method of claim 46, further comprising reading different values for the coefficient k0A from a memory unit for different types of dialyzers.

48. The method according to claim 41, further comprising measuring the clearance K or dialysance D at an initial blood flowrate Q_{b} and an initial dialysis fluid flowrate Q_{d}, and calculating the coefficient k0A.

49. A computer software product for performing the method of claim 41.

50. A computer software product for performing the method of claim 38.
